(19) United States
(12) Patent Application Publication
(10) Pub. No.: US 2022/0071940 A1
(43) Pub. Date: Mar. 10, 2022

(54) Antimicrobial Composition

(71) Applicant: Dutch Renewable Energy B.V., Muiden (NL)

(72) Inventor: Hubert Clemens Pellikaan, Utrecht (NL)

(21) Appl. No.: 17/528,444

(22) Filed: Nov. 17, 2021

Related U.S. Application Data
(62) Division of application No. 14/425,478, filed on Mar. 3, 2015.

(30) Foreign Application Priority Data
Sep. 3, 2012 (NL) 2009407

(51) Int. Cl. A61K 31/20; A61K 31/194; A61K 31/19; A61K 31/352; A61K 31/047; A61K 31/723

(52) U.S. Cl. CPC A61K 31/20; A61K 31/194; A61K 31/19; Y02A 50/30; A61K 31/047; A61K 31/723; A61K 31/352

(57) ABSTRACT

Provided herein is a method of treating an infectious disease by administering to a patient a pharmaceutical formulation dissolved in aqueous medium having at least one free fatty acid, or a derivative and/or a pharmaceutically acceptable salt of the same, where the free fatty acid has from 6 to 16 carbon atoms, at least one carboxylic acid or a pharmaceutically acceptable salt of the same, and/or at least one carbohydrate or a pharmaceutically acceptable salt of the same. The carbohydrate may be selected from a hydrogenated carbohydrate, a monosaccharide, a disaccharide, a polysaccharide or a combination of the same. The antimicrobial composition provides enhanced anti-adhesion and/or disinfecting properties. The composition may be formulated into a drink, gel or spray, suitable for treating or preventing the first stage in pathogenesis.

### CROSS REFERENCE TO RELATED APPLICATIONS

This application is a divisional of U.S. patent application Ser. No. 14/425,478 filed on Mar. 3, 2015, which is the United States national phase of International Application No. PCT/NL2013/050628 filed Aug. 30, 2013, and claims priority to The Netherlands Patent Application No. 2009407 filed Sep. 3, 2012, the disclosures of which are hereby incorporated in their entirety by reference.

### BACKGROUND OF THE INVENTION

### Field of the Invention

The present invention relates to an antimicrobial composition and a pharmaceutical formulation comprising the antimicrobial composition, a pharmaceutical formulation for treating or preventing the first stage in pathogenesis and the use of an organic acid as an anti-adhesion agent.

### Description of Related Art

The throat is an open crossroad in the respiratory tract as well as a reservoir for many pathogens. A sore throat can be the starting point of many infections of the upper respiratory tract, e.g. otitis media and sinusitis, and the lower respiratory tract, e.g. bronchitis and pneumonia. In addition to the throat, also wounds can be a starting point of many infections. Adhesion and colonization of human tissue is known to be the first stage in pathogenesis of many infectious diseases.

A sore throat is initially mostly viral in nature, common viruses may provide an initial conditioning to the host mucosa that favours bacterial adherence which subsequently leads to bacterial biofilm formation. To overcome the adhesion of the pathogens to the host mucosa is a viable strategy in preventing infection. Additionally, the use of effective inhibitory agents may also exert a therapeutic advantage by inhibiting extended colonization and restricting the spread of infection. Causes of a sore throat vary from having a dry throat to severe throat infections like laryngitis. An effective composition must at least soothe the throat and take away the cause or causes of the sore throat.

### SUMMARY OF THE INVENTION

The present invention therefore aims to provide an antimicrobial composition for treating or preventing the first stage in pathogenesis in order to prevent infections.

### BRIEF DESCRIPTION OF THE DRAWINGS

FIG. 1 shows a microscopic picture of a well of a 6-well plate showing microorganism growth after treatment with composition A;

FIG. 2 shows a microscopic picture of a well of a 6-well plate showing microorganism growth after treatment with composition B;

FIG. 3 shows a microscopic picture of a well of a 6-well plate showing microorganism growth after treatment with composition C; and,

FIG. 4 shows a microscopic picture of a well of a 6-well plate showing microorganism growth after no treatment (positive control).

### DETAILED DESCRIPTION OF THE INVENTION

The present invention thereto provides an antimicrobial composition comprising at least one free fatty acid or its derivative and/or pharmaceutically acceptable salt thereof, at least one carboxylic acid or a pharmaceutically acceptable salt thereof and/or at least one carbohydrate or a pharmaceutically acceptable salt thereof. It was found that a composition comprising a combination of at least one free fatty acid and at least one carboxylic acid having excellent disinfecting properties, i.e. exhibiting antiviral and antibacterial properties. It was further found that the combination of at least one free fatty acid and at least one carbohydrate, optionally in combination with at least one carboxylic acid, exerts a dual antimicrobial effect. The carbohydrate as well as the free fatty acid block the adhesion of the bacterial cells to the throat tissue and therefore exhibit good anti-adhesion properties. The free fatty acid and carboxylic acid exerts a potent microbicidal effect on contact with a microbial cell by dissolution of the lipid components of the microbial cell membrane.

The free fatty acids may be selected from saturated free fatty acid, unsaturated free fatty acid and combinations thereof, having from 4 to 22 carbon atoms, because free fatty acids having a chain length from 4 to 22 carbon atoms show to have antiviral and antibacterial properties. Preferably the free fatty acid of the present invention having from 5 to 18 carbon atoms, more preferably the free fatty acid having from 6 to 16 carbon atoms. It was found that a free fatty acid having from 6 to 16 carbon atoms is well soluble. Even further it was found that free fatty acids with chain lengths varying from 6 to 16 carbon atoms are well soluble and exhibits good anti-adhesion properties and exerts potent microbicidal effect on contact with a microbial cell in case the free fatty acids are solubilised in a medium having a pH in the range of about 5.5 to about 7.0. Even more preferred the free fatty acid of the present invention having from 7 to 14 carbon atoms. Most preferred the free fatty acid having from 8 to 10 carbon atoms because free fatty acids with chain lengths varying from 8 to 10 carbon atoms show to have more strongly antiviral and antibacterial properties, i.e. inhibition of bacterial growth, compared to free fatty acids with chain lengths outside the most preferred range.

Derivatives of free fatty acids used in the antimicrobial composition of the present invention may be selected from esters of free fatty acids, including lipids such as C₆-C₁₆ monoglyceride, diglyceride, triglyceride or combinations thereof. Typically, lipids are preferred including C₈-C₁₀ monoglyceride, e.g. caprylic acid monoglyceride.

Preferred free fatty acids suitable for use in the antimicrobial composition of the present invention are selected from enanthic acid, caproic acid, caprylic acid, pelargonic acid, capric acid, undecanoic acid, undecylenic acid, lauric acid, myristic acid, palmitic acid and combinations thereof, and pharmaceutically acceptable salts and esters thereof. More preferred the free fatty acid of the present invention is selected from caprylic acid, pelargonic acid, capric acid, undecanoic acid, lauric acid and combinations thereof. Most preferred the free fatty acid of the present invention is selected from caprylic acid, capric acid and combinations thereof, and pharmaceutically acceptable salts and esters thereof, due to the more strongly antiviral and antibacterial properties compared to the other free fatty acids.

In addition to the antiviral and antibacterial properties of the free fatty acids of the present invention it was also found that the free fatty acids exhibit anti-adhesion properties. In particular free fatty acids selected from caprylic acid, capric acid and combinations thereof exhibit good anti-adhesion properties, i.e. inhibition adhesion of pathogenic bacteria to the human epithelium.

In an embodiment of the present invention, the antimicrobial composition of the present invention comprises in addition to at least one free fatty acid having from 6 to 16 carbon atoms a free fatty acid having less than 6 or more than 16 carbon atoms selected from valeric acid, stearic acid, oleic acid, linoleic acid, linolenic acid and combinations thereof.

The carboxylic acid may be any carboxylic acid exhibiting antiviral and antibacterial properties. In particular the carboxylic acid may be selected from alpha hydroxy acids, diacids, polyacids and combinations thereof. Preferably the carboxylic acid may be selected from glycolic acid, lactic acid, citric acid, mandelic acid, oxalic acid, malonic acid, succinic acid, glutaric acid, tartaric acid, adipic acid, pimelic acid, malic acid, maleic acid, fumaric acid and combinations thereof. It was found that the carboxylic acids present in the composition of the present invention exhibit excellent antiviral and antibacterial properties at a neutral pH, i.e. in the range of about 5.5 to about 7.0.

It was even further found that carboxylic acids exhibit excellent antiviral and antibacterial properties in low amounts within the range of about 0.01% to about 4% by weight of the total weight of the composition. Preferably, the concentration of the carboxylic acids is about 0.1% to about 3% by weight of the total weight of the composition, more preferred the concentration is about 0.2% to about 2% by weight of the total weight of the composition, even more preferred the concentration is about 0.3% to about 1% by weight of the total weight of the composition and most preferred the concentration is about 0.4% to about 0.5% by weight of the total weight of the composition. In a preferred embodiment, the carboxylic acid is selected from citric acid or a pharmaceutically acceptable salt thereof. It was found that a composition of the present invention comprising citric acid was effective against E. coli.

The carbohydrate used in the antimicrobial composition of the present invention is preferably selected from a hydrogenated carbohydrate, a monosaccharide, a disaccharide, a polysaccharide and combinations thereof. In particular, the carbohydrate is preferably selected from monosaccharides and/or disaccharides including for example glucose, fructose, galactose, ribose, sucrose, maltose, lactose, cellulose, starch, glycogen, xylitol, mannitol, sorbitol, erythritol, mannose, D-mannose and combinations thereof. It was found that monosaccharides and/or disaccharides exhibit good anti-adhesion properties. More preferably the carbohydrates used in the present invention are selected from sucrose, xylitol, mannitol and combinations thereof exhibit excellent anti-adhesion properties.

In one embodiment of the present invention, the antimicrobial composition comprises a free fatty acid being caprylic acid and a carbohydrate being xylitol. It was found that the free fatty acid e.g. caprylic acid, exhibits clear anti-adhesion properties and that in combination with the carbohydrate, e.g. xylitol, an additional effect is provided on the adhesion inhibition.

In a further embodiment of the present invention, the antimicrobial composition comprises a carbohydrate selected from monosaccharides and/or disaccharides in combination with a carbohydrate selected from hydrogenated carbohydrates and/or polysaccharides, e.g. dextran, dextran sulphate, hyaluron, mannan, manuka honey and combinations thereof. It was found that a combination of two or more carbohydrates selected from a monosaccharide, e.g. xylitol, and polysaccharides, e.g. manuka honey and dextran, exhibits excellent anti-adhesion properties. In one embodiment of the present invention, the antimicrobial composition comprises a free fatty acid being caprylic acid and carbohydrates being xylitol, manuka honey, dextran or combinations thereof. It was found that the free fatty acid, e.g. caprylic acid, exhibits clear anti-adhesion properties and that in combination with the carbohydrates, e.g. xylitol and dextran, an additional effect is provided on the adhesion inhibition.

In another embodiment of the present invention, the antimicrobial composition further comprising at least one flavonoid or a pharmaceutically acceptable salt thereof. Preferably the flavonoid is selected from flavanols, flavones, anthocyanidins, isoflavonoids, neoflavonoids and combinations thereof. Most preferred the flavonoid is a proanthocyanidin. Also flavonoid-comprising components might be used. An example of a flavonoid-comprising component is Echinacea Purpurea extract.

The antimicrobial composition may further comprise at least one flavouring agent, viscosity-enhancing agent, emulsifying agent, acidity-regulating agent, humectant and/or preservative. The amount of additional agents used is preferably restricted, because it was found that the addition of several agents reduces the antimicrobial activity of the antimicrobial composition.

The antimicrobial composition may comprise at least one flavouring agent. Preferably the flavouring agent is selected from peppermint oil, candy flavour, sodium cyclamate and combinations thereof. The antimicrobial composition of the present invention can be used without comprising a flavouring agent.

The antimicrobial composition may comprise at least one viscosity-enhancing agent, i.e. thickening agent. Preferably the viscosity-enhancing agent is selected from xanthan gum, alginic acid, agar, carrageenan, locust bean gum, pectin, cellulose derivatives, gelatin and combinations thereof.

The antimicrobial composition may comprise at least one emulsifying agent, e.g. surfactant. Preferably the emulsifying agent is selected from polysorbate (Tween) 20, polysorbate 40, polysorbate 60, polysorbate 80, polyoxyethylene glycol alkyl ethers, glucoside alkyl ethers, polyoxyethylene glycol octylphenol ethers, polyoxyethylene glycol alkylphenol ethers, glycerol alkyl esters, poloxamers, polyoxyl castor oil, and combinations thereof. More preferably the emulsifying agent is polysorbate 80.

The antimicrobial composition may comprise at least one acidity-regulating agent, i.e. pH control agent. Preferably the acidity-regulating agent is selected from lactic acid, acetic acid, citric acid, adipate, tartaric acid, malic acid, fumaric acid and combinations thereof.

The antimicrobial composition may comprise at least one humectant, i.e. a desiccant. Preferably the humectant is selected from propylene glycol, hexylene glycol, butylene glycol, glyceryl triacetate, vinyl alcohol, neoagarobiose, sugar polyols such as glycerol, sorbitol, xylitol, xanthan gum and maltitol, polymeric polyols like polydextrose, quillaia, lactic acid, urea, glycerine, aloe vera gel, MP Diol, alpha hydroxy acids like lactic acid, honey and combinations thereof.

Preservatives may be used to prevent decomposition and to ensure microbiologic stability. Preservatives may be selected from benzethonium chloride, benzisothiazolinone, biphenyl, tert-butylhydroquinone, calcium tartrate, cetylpyridinium chloride, chloroacetamide, choji oil, dimethyl dicarbonate, ethylenediaminetetraacetic acid, germaben, glycolic acid, hexamethylenetetramine, imidazolidinyl urea, iodopropynyl butylcarbamate, isothiazolinone, lactic acid, methylchloroisothiazolinone, methylisothiazolinone, natamycin, nisin, parabens, persulfate, 2-phenylphenol, phytic acid, polyaminopropyl biguanide, benzoate salts, e.g. sodium benzoate, sorbate salts, e.g. potassium sorbate, chloride salts, e.g. sodium chloride, nitrate salts, e.g. sodium nitrate, quaternium-15, sorbic acid, sulfur dioxide, tiabendazole and combinations thereof.

In one embodiment of the present invention the components of the antimicrobial composition are dissolved in an aqueous medium. Preferably the aqueous medium has a pH in the range of about 5.5 to about 7.0. Most preferred the aqueous medium has a pH in the range of about 6.0 to about 6.5. It was found that the free fatty acids of the present invention are sufficiently soluble in the aqueous medium having a pH in the range of about 6.0 to about 6.5 and wherein the free fatty acids, optionally in combination with the carboxylic acids, exhibits antiviral and antibacterial properties. An aqueous medium having a pH lower than about 5.5 is no longer suitable for solubilising the free fatty acids of the present invention. It was found that an aqueous medium having a pH higher than about 7.0 results in an antimicrobial composition with reduced antiviral and antibacterial properties.

The term “about” as used herein is intended to include values, particularly within 10% of the stated values.

In one embodiment of the present invention, the antimicrobial composition as described above is dissolved in an aqueous medium substantially free of multivalent metal ions, e.g. divalent metal ions such as calcium and magnesium ions, having a pH of about 6.0 to about 6.5. In another embodiment of the present invention, the antimicrobial composition as described above is at least partially dissolved in an aqueous medium comprising multivalent metal ions, e.g. calcium ions, having a pH of about 6.0 to about 6.5. In the presence of multivalent metal ions some of the free fatty acids used in the antimicrobial composition of the present invention precipitate in the form of a multivalent metal ion-free fatty acid complex. The complex formed still exhibits antiviral and antibacterial properties.

The antimicrobial composition of the present invention may further comprise sequestrants complexing the metal ions, e.g. calcium ions, from the solution. Preferably sequestrants may be selected from calcium disodium ethylene diamine tetra-acetate, glucono delta-lactone, sodium gluconate, potassium gluconate, sodium tripolyphosphate, sodium hexametaphosphate and combinations thereof.

It was further found that the amount of surfactants in the antimicrobial composition must be as low as possible to maximize the antiviral and antibacterial properties of the free fatty acid. Preferably the amount of surfactants is less than 10% by weight of the total weight of free fatty acid present in the antimicrobial composition. More preferably the amount of surfactants is less than 8% by weight, less than 6% by weight. Most preferred the amount of surfactant is about 4% by weight of the total weight of free fatty acid present in the antimicrobial composition.

In another aspect of the present invention, a pharmaceutical formulation comprising the antimicrobial composition of the present invention is provided. Preferably the pharmaceutical formulation is in the form of a drink, gel, drops or spray. Even more preferred the pharmaceutical formulation is in the form of a throat spray, mouthwash, nasal spray, eardrops, wound spray or wound gel. The antimicrobial composition may also be formulated in the form of a syrup.

In even another aspect of the present invention, a pharmaceutical formulation comprising the antimicrobial composition of the present invention is provided for treating or preventing the first stage in pathogenesis in order to prevent infections. Preferably the pharmaceutical formulation comprising the antimicrobial composition of the present invention is provided for treating or preventing a sore throat and, as a consequence, preventing respiratory tract infections. Also preferred the pharmaceutical formulation comprising the antimicrobial composition of the present invention is provided for treating or preventing otitis media or outer ear infection. The pharmaceutical formulation comprising the antimicrobial composition of the present invention is provided for treating or preventing wound infection diseases.

In particular a pharmaceutical formulation comprising the antimicrobial composition of the present invention is provided for preventing or reducing the adhesion of bacteria and other microorganism selected from gram-positive bacteria, e.g. Staphylococcus aureus, β-hemolytic Streptococcus including group A. Streptococcus pyogenes, y-hemolytic Streptococcus including group C. Streptococcus faecalis and/or α-hemolytic Streptococcus including Streptococcus pneumoniae, gram-negative bacteria, e.g. Escherichia coli, Enterobacter aerogenes, Enterobacter and/or Haemophilus influenzae, Candida albicans and combinations thereof.

The pharmaceutical formulation comprising the antimicrobial composition of the present invention may also be used for the treatment of second and additional stages in the pathogenesis and/or treating multiple pathogenetical processes simultaneously.

In an even further aspect of the present invention, the present invention relates to the use of a free fatty acid as an anti-adhesion agent wherein the free fatty acid is selected from a free fatty acid having from 6 to 16 carbon atoms. The free fatty acid having from 6 to 16 carbon atoms may be preferably selected from caprylic acid, capric acid and combinations thereof, since it was found that both free fatty acids exhibits excellent anti-adhesion properties. However, the free fatty acid may also be selected from enanthic acid, caproic acid, caprylic acid, pelargonic acid, capric acid, undecanoic acid, undecylenic acid, lauric acid, myristic acid, palmitic acid and combinations thereof, and pharmaceutically acceptable salts and esters thereof.

The invention will now be further illustrated with reference to the following examples.

### EXAMPLES

4 different compositions were produced. The ingredients and amounts of the 4 different compositions are provided in table 1. Caprylic acid was dissolved in 50 kg water and the solution was adjusted to a pH of 6.5 by adding a mixture of lactic acid (90%) and sodium hydroxide (20%). Some precipitation of caprylic acid was allowed. The mixture formed was used as composition C.

TABLE 1
Compositions including weight-%

| Composition | A | B | C |
|---|---|---|---|
| Sodium Caprylate | 1.27 | 1.27 | 1.27 |
| Caprylic acid | — | — | — |
| Xylitol | 10.00 | 10.00 | — |
| Dextran 40 kD | 1.00 | 1.00 | — |
| Manuka Honey | 0.10 | 0.10 | — |
| Whey protein | — | — | — |
| Proanthocyanidin | 0.04 | — | — |
| Flavouring agent | 0.04 | 0.04 | — |
| Sodium cyclamate | 0.8 | 0.8 | — |
| Xanthan gum | 0.25 | 0.25 | — |
| Polysorbate 80 | 0.05 | 0.05 | — |
| Sodium benzoate | 0.04 | 0.04 | — |
| Potassium sorbate | 0.15 | 0.15 | — |
| Desiccant | — | — | — |
| Lactic acid/sodium hydroxide | qs ad pH 6.5 | qs ad pH 6.5 | qs ad pH 6.5 |
| Water | ad 100 | ad 100 | ad 100 |

For the preparation of compositions A and B, 750 L water was added to the caprylic acid solution and the mixture was stirred. A separate mixture of 10 kg comprising xylitol, dextran 40 kD, manuka honey, sodium cyclamate, xanthan gum, sodium benzoate and potassium sorbate was blended and added to the mixture and stirred for 10 minutes. 53 kg of xylitol was added and the bulky mixture was stirred for an additional 15 minutes. Additionally, a separate mixture of proanthocyanidin (only composition A), flavouring agent and polysorbate 80 was mixed. 3 L of the bulky mixture was added to the flavouring agent-polysorbate 80 mixture and firmly stirred. The firmly stirred suspension is added to the bulky mixture and the mixture was stirred for 15 minutes. The pH of the mixture was checked and if necessary adjusted with lactic acid (90%) or sodium hydroxide (20%) to a pH of 6.5.

3 ml of composition A was transferred into a well of a polystyrene 6-wells plate. The 6-wells plate was incubated for 30 min at 35-37° C. while shaking (±150 rpm). Subsequently, composition A was removed from the well and 3 ml 10⁵-10⁶ cfu/ml microorganism (Candida Albicans ATCC 10231) suspension was added to the well. The microorganisms were allowed to adhere for 2 h at 35-39° C. while shaking (±150 rpm). The well was washed twice with water to removed non-adhere cells. The adhered cells were stained with 3 ml crystal violet solution. The well was washed three times with water to remove excess of the crystal violet solution. The adhered cells were examined using a microscope.

The above-described procedure was repeated for composition B and composition C.

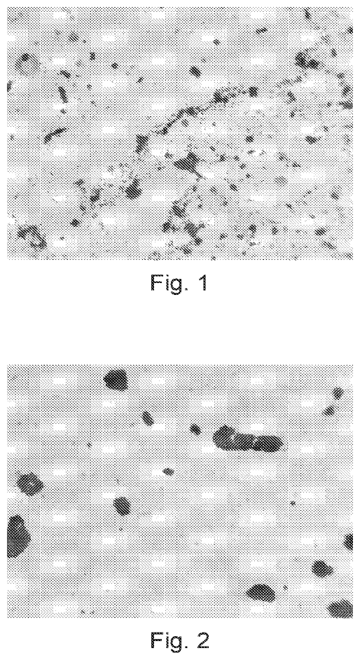

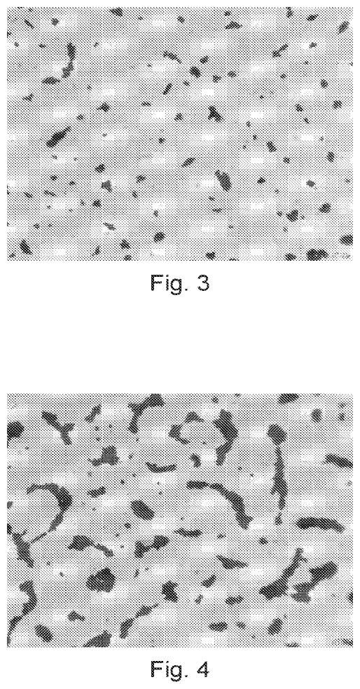

The percentage black on microscopic pictures taken from a bottom of a well treated with composition A (FIG. 1), composition B (FIG. 2) or composition C (FIG. 3) was compared to the percentage black on microscopic pictures taken from the bottom of a non-treated well (positive control; FIG. 4). Table 2 provides an overview of the comparative study.

TABLE 2
Comparative study adhesion of C. Albicans ATCC 10231

| | Average % black on | Inhibition of adhesion |
|---|---|---|
| | microscopic pictures of adhered | compared to positive |
| Composition | C. albicans ATCC 10231* | control (%) |
| Positive control | 10.86 | — |
| A | 4.42 | 59.3 |
| B | 4.02 | 63.0 |
| C | 5.98 | 44.9 |

*Average from three microscopic pictures

Compositions A and B were also tested against Streptococcus pyogenes ATCC 19615 and Staphylococcus aureus ATCC 6538. Table 3 and 4 provide an overview of the results of the comparative study.

In order to test the antibacterial effect of Compositions A and B, sterile flasks were filled aseptically with 10 g of Compositions A or B. The flasks are inoculated with Streptococcus pyogenes ATCC 19615 or Staphylococcus aureus ATCC 6538 to give a final microbial suspension of 10⁵ to 10⁶ cfu per mL.

The flasks are stored at 20-25° C. and at t=0 and 1 h, 1 g samples are taken from each flask to determine the number of viable microorganisms by plate count method. The results are provided in table 3 and 4.

TABLE 3
Comparative study microbicidal effect compositions
A and B against S. pyogenes

| | CFU count (¹⁰log) | | |
|---|---|---|---|
| Composition | t = 0 | t = 1 hour | ¹⁰log reduction |
| A | 5.9 | <1.0 | 4.9 |
| B | 5.9 | 2.1 | 3.9 |

TABLE 4
Comparative study microbicidal effect compositions
A and B against S. aureus

| | CFU count (¹⁰log) | | |
|---|---|---|---|
| Composition | t = 0 | t = 1 hour | ¹⁰log reduction |
| A | 5.4 | 2.8 | 2.6 |
| B | 5.4 | 3.2 | 2.2 |

Furthermore, the antibacterial effect of compositions comprising combinations of free fatty acids and carboxylic acids and derivatives were tested as well against Streptococcus pyogenes ATCC 19615 and Staphylococcus aureus ATCC 6538. The compositions D, E, F and G (see: table 5) were prepared by a similar method as given above. The pH of the compositions was adjusted to 6.0. The test results showed that compositions D, E, F and G exhibit antibacterial properties against S. pyogenes and S. aureus.

TABLE 5
Compositions including weight-%

| Composition | D | E | F | G |
|---|---|---|---|---|
| Caprylic acid | 1.00 | 1.00 | 4.00 | 2.00 |
| Caprylic acid monoglyceride | 1.00 | — | — | — |
| Sodium citrate | 1.00 | 1.00 | 4.00 | 4.00 |
| Eugenol | — | — | 0.10 | — |
| Flavouring agent | 0.50 | 0.50 | 0.50 | 0.50 |
| Sodium cyclamate | 0.80 | 0.80 | 0.80 | 0.80 |
| Xanthan gum | 0.25 | 0.25 | 0.25 | 0.25 |
| Sodium benzoate | 0.12 | 0.12 | 0.12 | 0.12 |
| Sodium hydroxide | 0.28 | 0.28 | 1.11 | 0.28 |
| Water | 95.06 | 96.06 | 89.12 | 92.06 |

In addition the antibacterial effect of compositions H, I and J (see: table 6) comprising combinations of free fatty acids and carboxylic acids were further tested against S. aureus, E. aerogenes, E. coli, C. albicans and A. brasiliensis. The results are shown in table 7 indicating the excellent antibacterial effect of the compositions.

TABLE 6
Compositions including weight-%

| Composition | H | I | J |
|---|---|---|---|
| Sodium Caprylate | 1.27 | 1.27 | 1.27 |
| Citric acid | 0.2 | 0.1 | 0.4 |
| Xylitol | 10.00 | 10.00 | 10.00 |
| Dextran 40 kD | 1.00 | 1.00 | 1.00 |
| Manuka Honey | 0.10 | 0.10 | 0.10 |
| Flavouring agent | 0.04 | 0.04 | 0.04 |
| Sodium cyclamate | 0.8 | 0.8 | 0.8 |
| Xanthan gum | 0.25 | 0.25 | 0.25 |
| Polysorbate 80 | 0.05 | 0.05 | 0.05 |
| Sodium benzoate | 0.04 | 0.04 | 0.04 |
| Potassium sorbate | 0.15 | 0.15 | 0.15 |
| Lactic acid/sodium hydroxide | qs ad pH 6.5 | qs ad pH 6.5 | qs ad pH 6.5 |
| Water | ad 100 | ad 100 | ad 100 |

TABLE 7
Microbicidal effect of compositions H,
I and J against various microorganisms

| | CFU count | | | | | | |
|---|---|---|---|---|---|---|---|
| | | H | | I | | J | |
| | t = | t = | t = | t = | t = | t = | t = |
| Microorganism | 0 | 1 h | 24 h | 1 h | 24 h | 1 h | 24 h |
| S. aureus | 10⁶ | <10 | <10 | <10 | <10 | <10 | <10 |
| E. aerogenes | 10⁶ | <10 | <10 | <10 | <10 | <10 | <10 |
| E. Coli | 10⁶ | <10 | <10 | <10 | <10 | <10 | <10 |
| C. albicans | 10⁶ | <10 | <10 | <10 | <10 | <10 | <10 |
| A. brasiliensis | 10⁶ | <10 | <10 | <10 | <10 | <10 | <10 |

The invention claimed is:

1. A method of treating an infectious disease comprising administering to a patient a pharmaceutical formulation comprising an antimicrobial composition dissolved in an aqueous medium having a pH between 6.0 and 7.0, the antimicrobial composition consisting of:
a) at least one free fatty acid or a pharmaceutically acceptable salt thereof, wherein the at least one free fatty acid has from 6 to 10 carbon atoms;
b) citric acid or the pharmaceutically acceptable salt thereof; and
c) a carbohydrate selected from xylitol, Manuka honey, dextran, and combinations thereof,
wherein a combination of components a), b), and c) achieves the antimicrobial composition which is effective in providing antimicrobial properties to the pharmaceutical formulation, and the component a) is sufficiently dissolved in the aqueous medium.

2. The method of claim 1, wherein the pharmaceutical formulation prevents adhesion of a microorganism to human tissue, wherein the microorganism is in gram-positive bacteria, gram-negative bacteria, and/or other microorganism to human tissue.

3. The method according to claim 2, wherein the gram-positive bacteria is selected from the group consisting of Staphylococcus aureus, Streptococcus pyogenes and/or Streptococcus faecalis, the gram-negative bacteria is selected from the group consisting of Escherichia coli, Enterobacter aerogenes, Enterobacter, and/or Haemophilus influenzae and/or the other microorganism is Candida albicans.

4. The method according to claim 1, wherein the at least one free fatty acid has from 7 to 10 carbon atoms and is selected from a group consisting of saturated free fatty acid, unsaturated free fatty acid, and combinations thereof.

5. The method according to claim 1, wherein the at least one free fatty acid is selected from a group consisting of caprylic acid, capric acid, combinations thereof, and pharmaceutically acceptable salts and esters thereof.

6. The method according to claim 1, wherein the carbohydrate is a monosaccharide and/or a disaccharide.

7. The method according to claim 1, wherein the carbohydrate is selected from a group consisting of glucose, fructose, galactose, ribose, sucrose, maltose, lactose, xylitol, mannitol, sorbitol, erythritol, and combinations thereof.

8. The method according to claim 1, wherein the carbohydrate is selected from a group consisting of sucrose, xylitol, mannitol, and combinations thereof.

9. The method according to claim 1, wherein the at least one free fatty acid is caprylic acid and the carbohydrate is xylitol.

10. The method according to claim 1, wherein the pharmaceutical formulation further comprises an at least one flavonoid or a pharmaceutically acceptable salt thereof.

11. The method according to claim 1, wherein the pharmaceutical formulation further comprises an at least one flavonoid selected from a group consisting of flavanols, flavones, anthocyanidins, isoflavonoids, neoflavonoids, and combinations thereof.

12. The method according to claim 11, wherein the at least one flavonoid is a proanthocyanidin.

13. The method according to claim 1, wherein the pharmaceutical formulation further comprises an at least one flavouring agent, viscosity-enhancing agent, emulsifying agent, acidity-regulating agent, humectant, and/or preservative.

14. The method according to claim 1, wherein the pharmaceutical formulation further comprises an emulsifying agent which is a surfactant and wherein the amount of the surfactant is less than 10% by weight of the total weight of free fatty acid present.

15. The method according to claim 1, wherein the aqueous medium has a pH in the range of about 6.0 to about 6.5.
